(19) United States
(12) Patent Application Publication
(10) Pub. No.: US 2023/0357935 A1
(43) Pub. Date: Nov. 9, 2023

(54) METHANE PRODUCTION SYSTEM AND METHANE PRODUCTION METHOD

(71) Applicant: NGK INSULATORS, LTD., Nagoya-shi (JP)

(72) Inventors: Hirofumi KAN, Nagoya-city (JP); Atsushi TORII, Handa-city (JP)

(21) Appl. No.: 18/354,123

(22) Filed: Jul. 18, 2023

Related U.S. Application Data
(63) Continuation of application No. PCT/JP2022/009406, filed on Mar. 4, 2022.

(30) Foreign Application Priority Data
Mar. 11, 2021 (JP) 2021-039697

(51) Int. Cl. C25B 3/03; C25B 15/021; C25B 1/04; C25B 15/08

(52) U.S. Cl. CPC C25B 3/03; C25B 15/021; C25B 1/04; C25B 15/08

(57) ABSTRACT

A methane production system includes a co-electrolysis/reforming cell and a control unit that controls operating temperatures of the co-electrolysis/reforming cell. The co-electrolysis/reforming cell includes a first electrode, a second electrode, and an electrolyte disposed between the first electrode and the second electrode. The co-electrolysis/reforming cell operates in either a co-electrolysis mode in which H₂ and CO are produced at the first electrode from CO₂ and H₂O, or a reforming mode in which CH₄ is produced at the first electrode from the H₂ and CO produced in the co-electrolysis mode. The control unit makes an operating temperature of the co-electrolysis/reforming cell in the reforming mode lower than an operating temperature of the co-electrolysis/reforming cell in the co-electrolysis mode.

### CROSS-REFERENCE TO RELATED APPLICATION

This is a continuation of PCT/JP2022/009406, filed Mar. 4, 2022, which claims priority from Japanese Application No. 2021-039697, filed Mar. 11, 2021 the entire contents of which are hereby incorporated by reference.

### TECHNICAL FIELD

The present invention relates to a methane production system and a methane production method.

### BACKGROUND ART

JP 2018-154864A discloses a solid oxide electrolysis cell (abbreviated as “SOEC” hereinafter) provided with a hydrogen electrode at which H₂O is electrolyzed, an electrolyte capable of transferring O²⁻, and an oxygen electrode at which O₂ is produced from O²⁻ transferred from the hydrogen electrode through the electrolyte.

JP 2019-175636A discloses that H₂ and CO can be produced by co-electrolyzing CO₂ and H₂O at the hydrogen electrode of an SOEC.

### SUMMARY

In order to utilize, as fuel, H₂ and CO produced from CO₂ and H₂O through co-electrolysis using an SOEC, it is effective to produce CH₄ from H₂ and CO using a reforming device.

However, in order to transport H₂ and CO from a plant where an SOEC is installed to a plant where a reforming device is installed, H₂ and CO need to be separated and liquefied.

Therefore, there is demand for performing production of H₂ and CO and production of CH₄ on-site (i.e., in one facility).

The present invention has been made in view of the above-described circumstances, and aims to provide a methane production system and a methane production method with which H₂, CO, and CH₄ can be produced on-site.

A methane production system according to the present invention includes a co-electrolysis/reforming cell and a control unit that controls operating temperatures of the co-electrolysis/reforming cell. The co-electrolysis/reforming cell includes a first electrode, a second electrode, and an electrolyte disposed between the first electrode and the second electrode. The co-electrolysis/reforming cell operates in either a co-electrolysis mode in which H₂ and CO are produced at the first electrode from CO₂ and H₂O, or a reforming mode in which CH₄ is produced at the first electrode from the H₂ and CO produced in the co-electrolysis mode. The control unit makes an operating temperature of the co-electrolysis/reforming cell in the reforming mode lower than an operating temperature of the co-electrolysis/reforming cell in the co-electrolysis mode.

According to the present invention, it is possible to provide a methane production system and a methane production method with which H₂, CO, and CH₄ can be produced on-site.

### BRIEF DESCRIPTION OF DRAWINGS

FIG. 1 is a block diagram showing a configuration of a methane production system.

FIG. 2 is a perspective view of a co-electrolysis/reforming device.

FIG. 3 is a cross-sectional view of the co-electrolysis/reforming device.

FIG. 4 is a perspective view of the co-electrolysis/reforming cell.

FIG. 5 is a cross-sectional view of the co-electrolysis/reforming cell.

FIG. 6 is a flowchart illustrating a methane production system.

### DESCRIPTION OF EMBODIMENTS

### Methane Production System

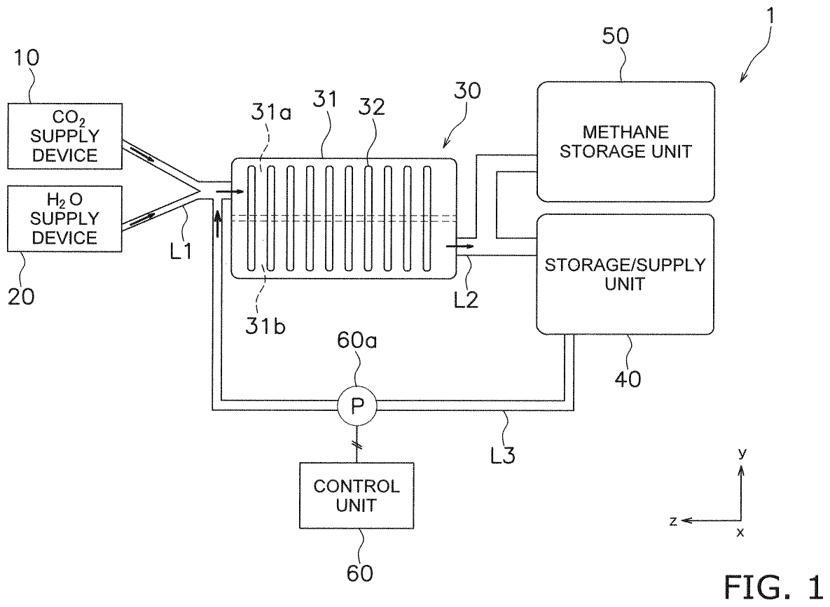

FIG. 1 is a block diagram showing a configuration of a methane production system 1 according to this embodiment.

The methane production system 1 includes a CO₂ supply device 10, an H₂O supply device 20, a co-electrolysis/reforming device 30, a storage/supply unit 40, a methane storage unit 50, and a control unit 60.

The CO₂ supply device 10 is connected to the co-electrolysis/reforming device 30 via a first pipe L1. When each co-electrolysis/reforming cell 32, which will be described later, operates in a co-electrolysis mode, the CO₂ supply device 10 supplies CO₂ (carbon dioxide) to the co-electrolysis/reforming device 30. It is preferable that the amount of CO₂ supplied from the CO₂ supply device 10 to the co-electrolysis/reforming device 30 is constant. Accordingly, it is possible to suppress the production of C (solid carbon) and CO₂ due to disproportionation reaction of CO (carbon monoxide) produced in the co-electrolysis/reforming cells 32 of the co-electrolysis/reforming device 30. As a result, it is possible to suppress deterioration of the electrode activity of the first electrode 2 of each co-electrolysis/reforming cell 32, which will be described later. When each co-electrolysis/reforming cell 32, which will be described later, operates in a reforming mode, the CO₂ supply device 10 does not supply CO₂ to the co-electrolysis/reforming device 30.

The H₂O supply device 20 is connected to the co-electrolysis/reforming device 30 via the first pipe L1. When each co-electrolysis/reforming cell 32 operates in the co-electrolysis mode, the H₂O supply device 20 supplies H₂O (water content) to the co-electrolysis/reforming device 30. The entirety or most of H₂O supplied from the H₂O supply device 20 to the co-electrolysis/reforming device 30 is gas (steam), but part of the H₂O may be liquid (water). When each co-electrolysis/reforming cell 32 operates in the reforming mode, the H₂O supply device 20 does not supply H₂O to the co-electrolysis/reforming device 30.

The co-electrolysis/reforming device 30 includes a manifold 31 and a plurality of co-electrolysis/reforming cells 32.

The manifold 31 has a configuration in which gas can be distributed to the co-electrolysis/reforming cells 32 and gas can be collected from the co-electrolysis/reforming device. The manifold 31 internally has a gas supply chamber 31a and a gas collection chamber 31b. The gas supply chamber 31a and the gas collection chamber 31b are airtightly separated from each other.

The first pipe L1 is connected to the gas supply chamber 31a. In the co-electrolysis mode, CO₂ and H₂O are supplied from the first pipe L1 to the gas supply chamber 31a. In the reforming mode, H₂ and CO are supplied from the first pipe L1 to the gas supply chamber 31a.

The gas collection chamber 31b collects gas produced in the co-electrolysis/reforming cells 32. A second pipe L2 is connected to the gas collection chamber 31b. In the co-electrolysis mode, H₂ and CO are discharged from the gas collection chamber 31b to the second pipe L2. In the reforming mode, CH₄ and H₂O are discharged from the gas collection chamber 31b to the second pipe L2. A reforming catalyst may be placed in the gas collection chamber 31b. The reforming catalyst may be in the form of pellets. The gas collection chamber 31b may be filled with the reforming catalyst. It is possible to use Ru/Al₂O₃, Ni/Al₂O₃, or the like as a reforming catalyst, for example.

A base end portion of each co-electrolysis/reforming cell 32 is supported by the manifold 31. A leading end portion of each co-electrolysis/reforming cell 32 is a free end. The number of co-electrolysis/reforming cells 32 is not particularly limited as long as it is 1 or more.

The co-electrolysis/reforming cells 32 operate in either the co-electrolysis mode in which H₂ (hydrogen), CO, O₂ (oxygen) are produced from CO₂ and H₂O, or the reforming mode in which CH₄ (methane) is produced from the H₂ and CO produced in the co-electrolysis mode. The term “co-electrolysis” used in this specification refers to production of H₂, CO, and O₂ by electrolyzing CO₂ and H₂O together. The term “reforming” used in this specification refers to production of CH₄ and H₂O from H₂ and CO.

Each co-electrolysis/reforming cell 32 operates at high temperatures (e.g., 600° C. to 850° C.) in the co-electrolysis mode. The operating temperatures of the co-electrolysis/reforming cells 32 in the co-electrolysis mode are preferably 700° C. or more and 850° C. or less. By setting the operating temperature thereof to 700° C. or more, the CO concentration can be increased due to thermodynamic equilibrium. By setting the operating temperature thereof to 850° C. or less, the amount of oxide ions flowing through the interconnector can be suppressed.

In the reforming mode, each co-electrolysis/reforming cell 32 operates at a temperature (e.g., 200° C. to 500° C.) lower than the operating temperature of the co-electrolysis/reforming cell 32 in the co-electrolysis mode. The operating temperatures of the co-electrolysis/reforming cells 32 in the reforming mode are preferably 350° C. or more and 400° C. or less. By setting the operating temperature thereof to 350° C. or more, it is possible to suppress the phase transformation of constituent materials of the support substrate 35 to carbonates. By setting the operating temperature thereof to 400° C. or less, it is possible to inhibit the produced CH₄ from being re-reformed to H₂ and CO.

The wording “operating temperature of the co-electrolysis/reforming cell 32” used in this specification refers to the temperature at the center in the longitudinal direction (X-axis direction) of the co-electrolysis/reforming cell 32. The operating temperature of each co-electrolysis/reforming cell 32 is controlled by the control unit 60.

In the co-electrolysis mode, CO₂ and H₂O are supplied from the gas supply chamber 31a to the co-electrolysis/reforming cells 32. In the co-electrolysis/reforming cells 32, H₂, CO, and O²⁻ (oxygen ions) are produced from CO₂ and H₂O at the first electrode 2, and O₂ is produced from O²⁻ at the second electrode 4. The produced H₂ and CO are collected into the gas collection chamber 31b and discharged from the second pipe L2.

In the reforming mode, H₂ and CO are supplied from the gas supply chamber 31a to the co-electrolysis/reforming cells 32. In the co-electrolysis/reforming cells 32, CH₄ and H₂O are produced from H₂ and CO at the first electrode 2. The produced CH₄ and H₂O are collected into the gas collection chamber 31b and discharged from the second pipe L2.

An example of a configuration of the co-electrolysis/reforming device 30 will be described later.

The storage/supply unit 40 is connected to the co-electrolysis/reforming device 30. In this embodiment, the storage/supply unit 40 is connected to the co-electrolysis/reforming device 30 via the second pipe L2 and the third pipe L3. Specifically, the storage/supply unit 40 is directly connected to the gas collection chamber 31b of the co-electrolysis/reforming device 30 by the second pipe L2, and directly connected to the gas supply chamber 31a of the co-electrolysis/reforming device 30 by the third pipe L3.

When the co-electrolysis/reforming cells 32 operate in the co-electrolysis mode, the storage/supply unit 40 stores H₂ and CO discharged from the gas collection chamber 31b via the second pipe L2. Therefore, the H₂ and CO produced in the co-electrolysis/reforming device 30 are directly stored in the storage/supply unit 40 without changing their compositions.

When the co-electrolysis/reforming cells 32 operate in the reforming mode, the storage/supply unit 40 supplies the stored H₂ and CO to the gas supply chamber 31a via the third pipe L3. Therefore, the H₂ and CO produced in the co-electrolysis/reforming device 30 are directly returned to the co-electrolysis/reforming device 30 without changing their compositions. When the co-electrolysis/reforming cells 32 operate in the reforming mode, the storage/supply unit 40 does not receive gas discharged from the co-electrolysis/reforming device 30.

The methane storage unit 50 is connected to the co-electrolysis/reforming device 30. In this embodiment, the methane storage unit 50 is connected to the co-electrolysis/reforming device 30 via the second pipe L2. Specifically, the methane storage unit 50 is directly connected to the gas collection chamber 31b of the co-electrolysis/reforming device 30 by the second pipe L2.

When the co-electrolysis/reforming cells 32 operate in the reforming mode, the methane storage unit 50 stores CH₄ and H₂O discharged from the gas collection chamber 31b via the second pipe L2. When the co-electrolysis/reforming cells 32 operate in the co-electrolysis mode, the methane storage unit 50 does not receive gas discharged from the co-electrolysis/reforming device 30.

The control unit 60 controls the operating temperature of each co-electrolysis/reforming cell 32. The control unit 60 can control the operating temperature of the co-electrolysis/reforming cell 32 by adjusting, for example, at least one of a current value, the amount of gas supplied to the first electrode 2, and the amount of gas supplied to the first electrode 2, which will be described later. In this embodiment, the control unit 60 makes the operating temperature of the co-electrolysis/reforming cell 32 in the reforming mode lower than the operating temperature of the co-electrolysis/reforming cell 32 in the co-electrolysis mode.

When the co-electrolysis/reforming cells 32 operate in the reforming mode, the control unit 60 drives a pump 60a arranged in the third pipe L3. As a result, the H₂ and CO stored in the storage/supply unit 40 are supplied to the gas supply chamber 31a of the co-electrolysis/reforming device 30 via the third pipe L3. When the co-electrolysis/reforming cells 32 operate in the co-electrolysis mode, the control unit 60 does not drive the pump 60a.

### Configuration of Co-Electrolysis/Reforming Device 30

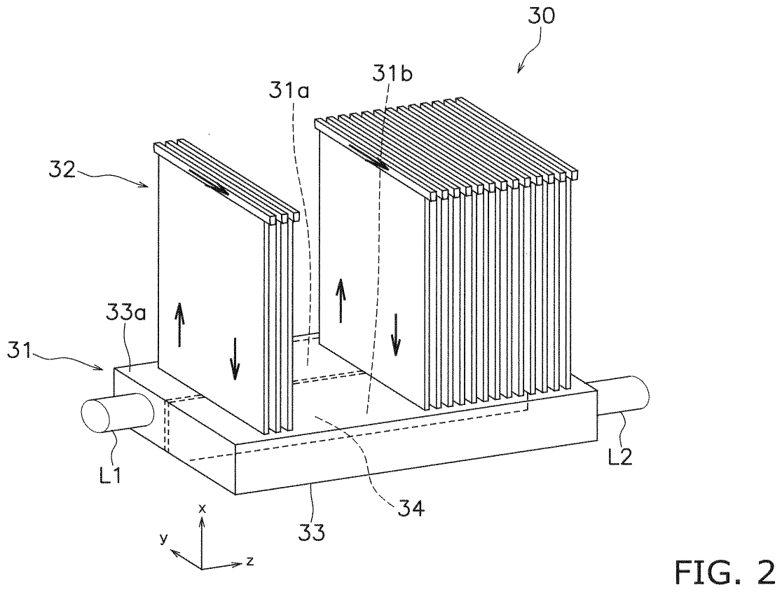

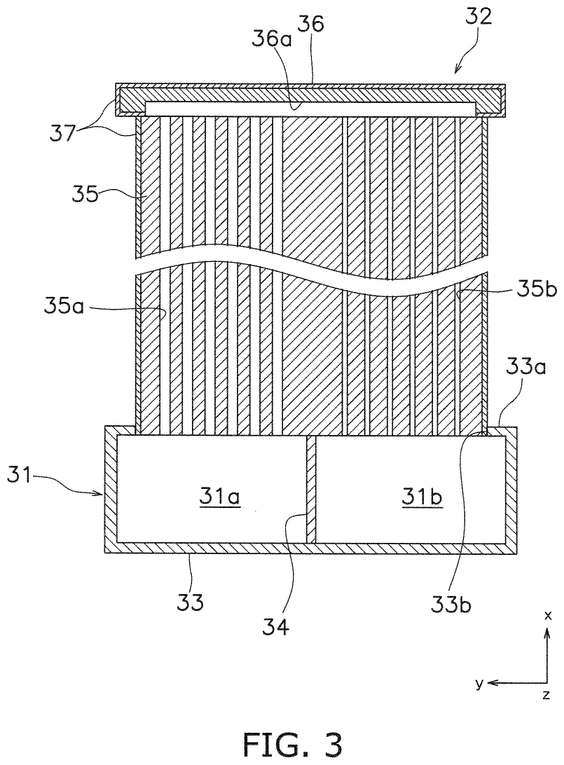

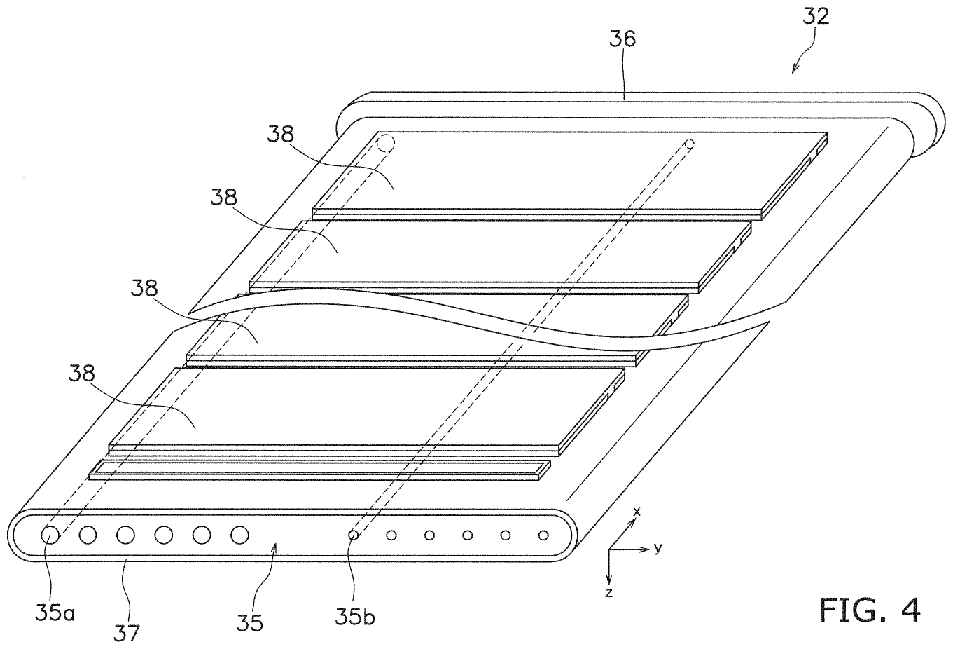

FIG. 2 is a perspective view of the co-electrolysis/reforming device 30. FIG. 3 is a cross-sectional view of the co-electrolysis/reforming device 30. FIG. 4 is a perspective view of the co-electrolysis/reforming device 32. Some of the co-electrolysis/reforming cells 32 are not shown in FIG. 2.

### Manifold 31

As shown in FIGS. 2 and 3, the manifold 31 includes a manifold main body portion 33 and a partition plate 34.

The manifold main body portion 33 is hollow. The partition plate 34 is arranged in the manifold main body portion 33. The partition plate 34 airtightly separates the gas supply chamber 31a and the gas collection chamber 31b from each other.

The manifold main body portion 33 has a top plate portion 33a. As shown in FIG. 3, the top plate portion 33a is provided with a plurality of through holes 33b. The through holes 33b are arranged side-by-side at predetermined intervals in the longitudinal direction (Z-axis direction) of the manifold main body portion 33. Each through hole 33b extends in the width direction (Y-axis direction) of the manifold main body portion 33. Although each through hole 33b is a long hole that is in communication with the gas supply chamber 31a and the gas collection chamber 31b in this embodiment, the through hole 33b may be divided into a hole that is in communication with the gas supply chamber 31a and a hole that is in communication with the gas collection chamber 31b.

### Co-Electrolysis/Reforming Cell 32

As shown in FIGS. 2 and 3, each co-electrolysis/reforming cell 32 extends in a direction away from the manifold 31. A base end portion of each co-electrolysis/reforming cell 32 is fixed to the through hole 33b of the top plate portion 33a using a bonding material (not shown) or the like. The base end portion of the co-electrolysis/reforming cell 32 may be inserted into the through hole 33b, or may protrude outward of the through hole 33b.

The co-electrolysis/reforming cells 32 are disposed such that their main surfaces face each other. The co-electrolysis/reforming cells 32 are arranged side-by-side at predetermined intervals along the longitudinal direction (Z-axis direction) of the manifold 31. That is, the arrangement direction of the co-electrolysis/reforming cells 32 extends in the longitudinal direction of the manifold 31. The co-electrolysis/reforming cells 32 are electrically connected in series or in a combination of series and parallel connections, using current collector members (not shown).

As shown in FIGS. 3 and 4, each co-electrolysis/reforming cell 32 includes a support substrate 35, a connection member 36, a coating layer 37, and a plurality of element portions 38. The co-electrolysis/reforming cell 32 according to this embodiment is a so-called horizontal-stripe type solid oxide electrolysis cell (SOEC).

The support substrate 35 is plate-shaped. In this embodiment, the vertical direction (X-axis direction) in FIG. 3 is the longitudinal direction of the support substrate 35, and the horizontal direction (Y-axis direction) in FIG. 3 is the width direction of the support substrate 35.

A plurality of first gas channels 35a and a plurality of second gas channels 35b are formed in the support substrate 35. The first gas channels 35a and the second gas channels 35b each extend from the base end portion to the leading end portion of the co-electrolysis/reforming cell 32 in the support substrate 35. The first gas channels 35a and the second gas channels 35b pass through the support substrate 35. The first gas channels 35a are disposed at intervals in the width direction of the support substrate 35. The second gas channels 35b are disposed at intervals in the width direction of the support substrate 35. Although the inner diameter of the first gas channels 35a is larger than the inner diameter of the second gas channels 35b in this embodiment, the inner diameter of the first gas channels 35a and the inner diameter of the second gas channels 35b are not particularly limited.

The first gas channels 35a are open to the gas supply chamber 31a. Gas flows into the first gas channels 35a from the gas supply chamber 31a. The second gas channels 35b are open to the gas collection chamber 31b. Gas flows out from the second gas channels 35b and enters the gas collection chamber 31b.

The support substrate 35 is made of a porous material having no electron conductivity so as to allow gas permeation while preventing short circuits between element portions 38. The support substrate 35 may be made of CSZ (calcia-stabilized zirconia), 8YSZ (yttria-stabilized zirconia), Y₂O₃(yttria), MgO (magnesium oxide), MgAl₂O₄ (magnesia alumina spinel), or a composite thereof, for example. The support substrate 35 may have a porosity of 20% to 60%. Note that the porosity mentioned in this specification is a value measured using the Archimedes' method.

The connection member 36 is attached to the leading end portion of the support substrate 35. The connection member 36 may be made of a porous material similar to that of the support substrate 35, for example. The connection member 36 internally has a connection channel 36a. The connection channel 36a is in communication with the first gas channels 35a and the second gas channels 35b.

The coating layer 37 covers outer surfaces of the support substrate 35 and the connection member 36. The coating layer 37 is denser than the support substrate 35 and the connection member 36. The coating layer 37 may have a porosity of about 0% to 7%. The coating layer 37 may be made of a material used in the later-described electrolyte 3, crystallized glass, or the like.

The element portions 38 are supported by the support substrate 35. The element portions 38 may be arranged on both main surfaces of the support substrate 35, or may be arranged on only one of the main surfaces.

Element portion 38

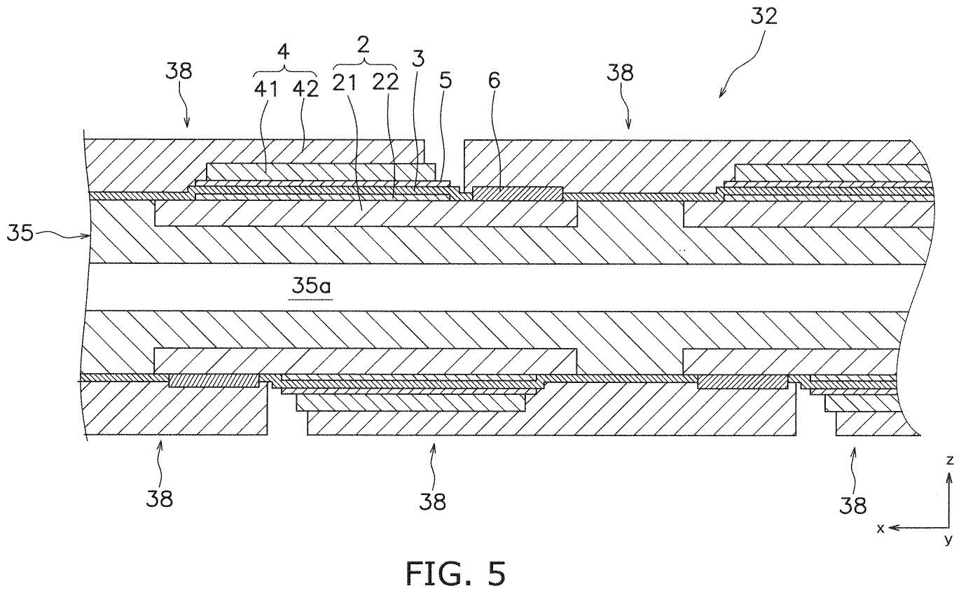

FIG. 5 is a cross-sectional view of the co-electrolysis/reforming cell 32 cut along the first gas channel 35a.

Each element portion 38 has a first electrode 2, an electrolyte 3, a second electrode 4, a reaction preventing film 5, and an interconnector 6.

When the co-electrolysis/reforming cell 32 operates in the co-electrolysis mode, at the first electrode 2, H₂, CO, and O²⁻ are produced from CO₂ and H₂O according to the chemical reaction of the co-electrolysis indicated by Chemical Equation (1) below.

First electrode2(co-electrolysis mode):CO₂+H₂O+4e⁻→CO+H₂+2O²⁻ (1)

When the co-electrolysis/reforming cell 32 operates in the reforming mode, at the first electrode 2, CH₄ and H₂O are produced from H₂ and CO according to the chemical reaction of the reforming indicated by Chemical Equation (2) below.

First electrode2(reforming mode):3H₂+CO→CH₄+H₂O (2)

The first electrode 2 has a first electrode base body 21 and a first electrode active portion 22.

The first electrode base body 21 is disposed on the support substrate 35. The first electrode base body 21 is embedded in a recess formed in a surface of the support substrate 35 in this embodiment, but may be placed on the surface of the support substrate 35. The first electrode base body 21 may have a thickness of 50 to 500 μm.

The first electrode base body 21 is made of a porous material having electron conductivity. The first electrode base body 21 preferably has higher electron conductivity than the first electrode active portion 22. The first electrode base body 21 optionally has oxygen ion conductivity. The first electrode base body 21 may be made of a composite of NiO and 8YSZ, a composite of NiO and Y₂O₃, a composite of NiO and CSZ, or the like, for example.

The first electrode active portion 22 is disposed on the first electrode base body 21. The first electrode active portion 22 may have a thickness of 5 to 100 μm. The first electrode active portion 22 has oxygen ion conductivity and electron conductivity. The first electrode active portion 22 preferably has higher oxygen ion conductivity than the first electrode base body 21. The first electrode active portion 22 may be made of a composite of NiO and 8YSZ, a composite of NiO and GDC (Ce, Gd)O₂ (gadolinium doped ceria), or the like, for example.

The electrolyte 3 is disposed between the first electrode 2 and the second electrode 4. The electrolyte 3 transfers O²⁻ produced at the first electrode 2 to the second electrode 4. The electrolyte 3 is disposed on the first electrode 2. In this embodiment, the electrolyte 3 extends in the longitudinal direction of the support substrate 35 between two interconnectors 6. The electrolyte 3 may have a thickness of 3 to 50 μm, for example.

The electrolyte 3 is made of a dense material that has oxygen ion conductivity and does not have electron conductivity. The electrolyte 3 is denser than the support substrate 35. The electrolyte 3 may have a porosity of 0% to 7%, for example. The electrolyte 3 may be made of 8YSZ, LSGM (lanthanum gallate), or the like, for example.

When the co-electrolysis/reforming cell 32 operates in the co-electrolysis mode, at the second electrode 4, O₂ is produced from O²⁻ transferred from the first electrode 2 through the electrolyte 3, according to the chemical reaction indicated by Chemical Equation (3) below.

Second electrode4(co-electrolysis mode):2O²⁻→O₂+4e⁻ (3)

When the co-electrolysis/reforming cell 32 operates in the reforming mode, the second electrode 4 is not particularly functional.

The second electrode 4 has a second electrode active portion 41 and a second electrode base body 42.

The second electrode active portion 41 is disposed on the reaction preventing film 5. The second electrode active portion 41 may have a thickness of 10 to 100 μm, for example.

The second electrode active portion 41 is made of a porous material having oxygen ion conductivity and electron conductivity. The second electrode active portion 41 preferably has higher oxygen ion conductivity than the second electrode base body 42. The second electrode active portion 41 may be made of LSCF=(La, Sr) (Co, Fe)O₃ (lanthanum strontium cobalt ferrite), LSF=(La, Sr) FeO₃ (lanthanum strontium ferrite), LNF=La(Ni, Fe)O₃ (lanthanum nickel ferrite), LSC=(La, Sr)CoO₃ (lanthanum strontium cobaltite), SSC=(Sm, Sr)CoO₃ (samarium strontium cobaltite), or the like, for example.

The second electrode base body 42 is disposed on the second electrode active portion 41. The second electrode base body 42 is electrically connected to the first electrode base body 21 of the adjacent element portion 38 via the interconnector 6. The second electrode base body 42 may have a thickness of 50 to 500 μm, for example.

The second electrode base body 42 is made of a porous material having electron conductivity. The second electrode base body 42 preferably has higher electron conductivity than the second electrode active portion 41. The second electrode base body 42 optionally has oxygen ion conductivity. The second electrode base body 42 may be made of LSCF, LSC, Ag (silver), Ag—Pd (silver palladium alloy), or the like, for example.

The reaction preventing film 5 is disposed between the electrolyte 3 and the second electrode active portion 41. The reaction preventing film 5 suppresses a reaction of substances contained in the electrolyte 3 and the second electrode active portion 41 to form a reaction layer having high electric resistance. The reaction preventing film 5 may have a thickness of 3 to 50 μm, for example. The reaction preventing film 5 is made of a dense material. The reaction preventing film 5 may be made of GDC, for example.

The interconnector 6 is connected to the second electrode base body 42 and the first electrode base body 21 of the adjacent element portion 38. The interconnector 6 may have a thickness of 10 to 100 μm, for example. The interconnector 6 is made of a dense material that have electron conductivity. The interconnector 6 is denser than the support substrate 35. The interconnector 6 may have a porosity of 0% to 7%. The interconnector 6 may be made of LaCrO₃ (lanthanum chromite), (Sr, La)TiO₃ (strontium titanate), or the like, for example.

### Methane Production Method

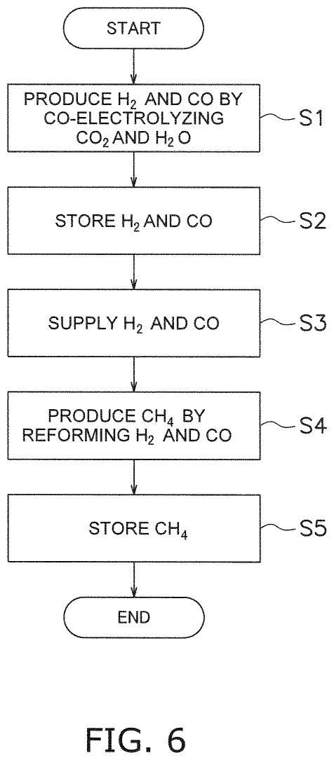

FIG. 6 is a flowchart illustrating a methane production method using the co-electrolysis/reforming cells 32.

In step S1, the co-electrolysis/reforming cells 32 produce H₂ and CO at the first electrode 2 by co-electrolyzing CO₂ and H₂O (co-electrolyzing step).

In step S2, the storage/supply unit 40 stores the H₂ and CO produced in the co-electrolysis/reforming cells 32 (first storing step).

In step S3, the storage/supply unit 40 supplies the stored H₂ and CO to the co-electrolysis/reforming cells 32 (supplying step).

In step S4, the co-electrolysis/reforming cells 32 produce CH₄ by reforming H₂ and CO (reforming step).

In step S5, the methane storage unit 50 stores the CH₄ produced in the co-electrolysis/reforming cells 32 (second storing step).

### Features

The methane production system 1 includes the co-electrolysis/reforming cells 32 and the control unit 60 that controls the operating temperatures of the co-electrolysis/reforming cells 32. The co-electrolysis/reforming cells 32 operate in either the co-electrolysis mode in which H₂ and CO are produced at the first electrode 2 from CO₂ and H₂O, or the reforming mode in which CH₄ is produced at the first electrode 2 from the H₂ and CO produced in the co-electrolysis mode. The control unit 60 makes the operating temperatures of the co-electrolysis/reforming cells 32 in the reforming mode lower than the operating temperatures of the co-electrolysis/reforming cells 32 in the co-electrolysis mode.

It is possible to produce CH₄ in the co-electrolysis/reforming cells 32 on-site, using the H₂ and CO produced in the co-electrolysis/reforming cells 32. Therefore, H₂ and CO do not need to be transported from a plant where a co-electrolysis device is installed to a plant where a reforming device is installed.

### Variation of Embodiment

Although an embodiment of the present invention has been described above, the present invention is not limited thereto, and various modifications can be made without departing from the spirit of the present invention.

### Variation 1

Although the co-electrolysis/reforming cell 32 is a horizontal-stripe type SOEC in the above embodiment, the co-electrolysis/reforming cell 32 is not limited to this. The co-electrolysis/reforming cell 32 may be a vertical-stripe type (hollow flat plate type), flat plate type, or cylindrical type SOEC, or the like. A configuration of a vertical-stripe type SOEC is described in JP 2015-125897A, for example. A configuration of a flat plate type SOEC is described in JP 2020-177839A, for example. A configuration of a cylindrical type SOEC is described in JP 2008-270203A, for example. However, horizontal-stripe type SOECs are particularly preferable because they have higher H₂O utilization efficiency than other SOECs.

### Variation 2

Although each element portion 38 has the first electrode 2, the electrolyte 3, the second electrode 4, the reaction preventing film 5, and the interconnector 6 in the above embodiment, it is sufficient that the element portion 38 includes at least the first electrode 2, the electrolyte 3, and the second electrode 4.

### Variation 3

When the co-electrolysis/reforming cells 32 operate in the reforming mode, the control unit 60 drives the pump 60a arranged in the third pipe L3 so as to supply H₂ and CO from the storage/supply unit 40 to the co-electrolysis/reforming device 30 in the above embodiment. However, the present invention is not limited to this. If pressure is applied to the H₂ and CO stored in the storage/supply unit 40, for example, the control unit 60 may adjust the opening degree of a flow control valve provided instead of the pump 60a so as to supply H₂ and CO from the storage/supply unit 40 to the co-electrolysis/reforming device 30.

### REFERENCE SIGNS LIST

1 Methane production system
10 CO₂ supply device
20 H₂O supply device
30 Co-electrolysis/reforming device
31 Manifold
32 Co-electrolysis/reforming cell
38 Element portion
2 First electrode
3 Electrolyte
4 Second electrode
40 Storage/supply unit
50 Methane storage unit
60 Control unit
L1 First pipe
L2 Second pipe
L3 Third pipe

1. A methane production system comprising:
a co-electrolysis/reforming cell having a first electrode, a second electrode, and an electrolyte disposed between the first electrode and the second electrode; and
a control unit configured to control an operating temperature of the co-electrolysis/reforming cell, wherein
the co-electrolysis/reforming cell operates in either a co-electrolysis mode in which H₂ and CO are produced at the first electrode from CO₂ and H₂O, or a reforming mode in which CH₄ is produced at the first electrode from the H₂ and CO produced in the co-electrolysis mode, and
the control unit makes an operating temperature of the co-electrolysis/reforming cell in the reforming mode lower than an operating temperature of the co-electrolysis/reforming cell in the co-electrolysis mode.

2. The methane production system according to claim 1, wherein
the operating temperature of the co-electrolysis/reforming cell in the co-electrolysis mode is 700° C. or more and 850° C. or less, and
the operating temperature of the co-electrolysis/reforming cell in the reforming mode is 350° C. or more and 400° C. or less.

3. The methane production system according to claim 1, further comprising
a storage/supply unit configured to store the H₂ and CO produced at the first electrode when the co-electrolysis/reforming cell operates in the co-electrolysis mode, and supply the stored H₂ and CO to the first electrode when the co-electrolysis/reforming cell operates in the reforming mode.

4. A methane production method using a co-electrolysis/reforming cell having a first electrode, a second electrode, and an electrolyte disposed between the first electrode and the second electrode, the method comprising:
a co-electrolyzing step of producing H₂ and CO at the first electrode from CO₂ and H₂O; and
a reforming step of producing CH₄ at the first electrode from the H₂ and CO produced in the co-electrolyzing step.

5. The methane production method according to claim 4, further comprising
a first storing step of storing the H₂ and CO produced in the co-electrolyzing step, wherein,
in the reforming step, CH₄ is produced from the H₂ and CO stored in the storing step.

6. The methane production method according to claim 5, further comprising
a second storing step of storing the CH₄ produced in the reforming step.
